(19) United States
(12) Patent Application Publication
(10) Pub. No.: US 2009/0023696 A1
(43) Pub. Date: Jan. 22, 2009

(54) USE OF C3-C10 17ALFA-ESTERS OF 9,11-DEHYDROCORTEXOLONE AS ANTI-GONADOTROPHIC AGENTS

(75) Inventors: Luigi Moro, Cairate (IT); Mauro Ajani, Lainate (Milano) (IT); Giuseppe Celasco, Genova (IT)

Correspondence Address: DARBY & DARBY P.C., P.O. BOX 770, Church Street Station, New York, NY, 10008-0770, US

(73) Assignee: COSMO BIO-TECHNOLOGIES SRL, Lainate (IT)

(21) Appl. No.: 12/066,754

(22) PCT Filed: Jun. 8, 2006
(86) PCT No.: PCT/EP2006/062995

§ 371 Date: May 20, 2008

(30) Foreign Application Priority Data
Sep. 14, 2005 (IT) MI2005A001695

(51) Int. Cl. A61K 31/56; A61P 15/00

(52) U.S. Cl. 514/177

(57) ABSTRACT

The present invention relates to the use of C₃-C₁₀ 17α-esters of 9,11-dehydrocortexolone as agents for inhibiting gonadotrophin secretion. The present invention therefore relates to the use of C₃-C₁₀ 17 α-esters of 9,11-dehydrocortexolone for the preparation of a medicine for the treatment of disorders associated with the secretion of gonadotrophin and with the corresponding pharmaceutical compounds. The present invention relates specifically to the use of 9,11-dehydrocortexolone 17α-butyrate.

The gonadotrophins (LH and FSH) are hormones produced in humans by the hypophysis (anterior pituitary gland), whose action is in turn controlled by the hypothalamus which releases gonadotrophin releasing hormone (GnRH) by pulsed secretion.

In turn, the gonadotrophins stimulate the male gonads to produce androgenic hormones, particularly testosterone.

Some skin diseases such as alopecia, hirsutism, seborrhoea, prostate diseases such as benign prostatic hypertrophy, prostate cancer, and other medical conditions such as polycystic ovary syndrome and precocious puberty are controlled by androgenic hormones and/or by gonadotrophins.

The treatment strategy for the aforesaid diseases is: i) to inhibit gonadotrophin secretion and consequently the production of androgenic hormones; ii) to block the conversion of androgens to their biologically active metabolites; iii) to interfere with the direct action of the androgens in the tissues (Motta M., in: The Medical Management of Prostate Cancer, L. Denis (ed.), pp 19-35. Springer-Verlag, Berlin—1988).

The first objective is normally achieved by using steroid hormones, or analogues and/or antagonists of the gonadotrophin releasing hormone (GnRH), both capable of suppressing gonadotrophin secretion and thus blocking the synthesis of hormones originating from the gonads.

The second objective is achieved by using androgen metabolism inhibitors which prevent the conversion of testosterone (T) to its principal metabolite, namely dihydrotestosterone (DHT). The third objective is achieved by the administration of substances, known as antiandrogens, which act as competitors at the receptors of the androgens, thus blocking the action of the androgens, regardless of their production site (testicles, ovaries, subrenal capsules).

In particular, the inhibition of hypophysial secretion of gonadotrophin is an extremely important mechanism in the treatment of pathologies such as tumours of the prostate, polycystic ovary syndrome, endometriosis, gonadotrophin-dependent precocious puberty, male contraception and aberrant sexual behaviour (Goodman & Gilman's The Pharmacological Basis of Therapeutics. 9^{th} Edition 1996, pp. 1379-1380. Ehrmann D. A., Polycystic ovary syndrome. N Engl J Med 2005; 352:1223-1236) and/or in all medical practice which requires modulation of gonadotrophin secretion, for example in fertilization procedures used to prevent hot flushes in women, and in ovarian stimulation in assisted fertility treatment (Ron-El A. et al., Induction of ovulation after GnRH antagonists. Human Reproduction Update 2000; 6:318-32).

International patent application WO03/014141 describes compounds belonging to the family of steroids structurally related to cortexolone (11-deoxycortisone) as having high antiandrogenic activity.

These compounds, such as cortexolone 17α-propionate, act by blocking the conversion of androgens into their biologically active metabolites, and/or by interfering with the direct action of the androgenic hormones in the tissues, but are not found to have any inhibitory effect on gonadotrophin section.

U.S. Pat. No. 3,780,177 describes a process for obtaining new fluorinated steroids, mentioning some cortexolone derivatives such as 9,11-dehydrocortexonole 17α-butyrate (diagram B) as intermediates of the synthesis.

During the evaluation of new steroids structurally related to the cortexolone family, it was surprisingly found that, in accordance with the object of the present

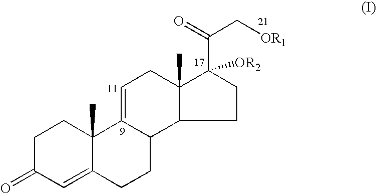

invention, C₃-C₁₀ 17α-esters of 9,11-dehydrocortexolone having the formula (I) in which R₁ is hydrogen and R₂ is a linear or branched C₃-C₁₀ acyl group show a powerful hypophysial activity, in addition to the characteristic antiandrogenic activity of the aforesaid family of substances; in fact, they act by significantly inhibiting gonadotrophin secretion. The present invention therefore relates to the novel use of C₃-C₁₀ 17α-esters of 9,11-dehydrocortexolone as anti-gonadotrophic agents.

The use of C₃-C₁₀ 17α-esters of 9,11-dehydrocortexolone according to the present invention is specifically intended for the preparation of compounds for the treatment of disorders closely related to excess gonadotrophin production, such as prostate tumour, polycystic ovary and gonadotrophin-dependent precocious puberty, for the control of aberrant sexual behaviour, for male contraception, and/or in all medical practice requiring modulation of gonadotrophin secretion, for example in the fertilization procedure used to prevent premature hot flushes in women and ovarian stimulation in assisted fertilization methods.

The present invention relates specifically to the use of 9,11-dehydrocortexolone 17α-butyrate of formula (II), corresponding to the compound of formula (I) in which R₁ is hydrogen and R₂ is a linear C₄ acyl,

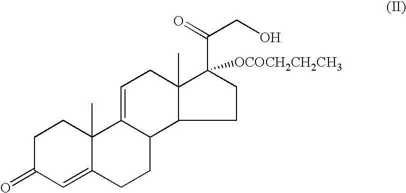

as an anti-gonadotrophic agent capable of inhibiting gonadotrophin secretion.

The present invention therefore also proposes the pharmaceutical compounds containing as their active principle a compound belonging to the family of the C₃-C₁₀ 17 α-esters of 9,11-dehydrocortexolone, particularly 9,11-dehydrocortexolone 17α-butyrate, in association with pharmacologically acceptable excipients and, optionally, any other active principles having a synergistic action.

The compounds of the invention can be formulated as injectable liquid pharmaceutical compounds such as monobasic or polyphase solutions and/or suspensions in solvents, or as solid pharmaceutical compounds such as tablets, capsules, pellets or liquid and/or semi-solid oral and/or topical pharmaceutical compounds such as solutions or suspensions, suppositories, globuli, vaginal suppositories, creams, ointments, lotions and/or gels, transdermal patches and sprays for cutaneous or nasal administration.

According to the present invention, the C₃-C₁₀ 17α-esters of 9,11-dehydrocortexolone, and particularly 9,11-dehydrocortexolone 17α-butyrate, can be administered systematically in quantities varying from 0.1 to 1000 mg per unit dose, and preferably from 0.5 to 500 mg. According to the present invention, the term “unit dose” refers to a single form of administration, such as a tablet, a capsule and/or an injection.

According to the present invention, the C₃-C₁₀ 17α-esters of 9,11-dehydrocortexolone, and particularly 9,11-dehydrocortexolone 17α-butyrate, can also be administered topically in quantities varying from 0.01 to 25%, and preferably from 0.01 to 2%, of the total weight of the formulation (cream, ointment, lotion, gel, transdermal patch and/or cutaneous spray).

The anti-gonadotrophic action was evaluated in an animal model by evaluation of the hypersecretion of the gonads induced by castration in parabiotic rats (Shipley E. G., in: Methods in Hormone Research, R. L Dorfman (ed.), vol. 2, pp 179-274. Academic Press, New York and London, 1962). The results of the inhibitory action of 9-dehydrocortexolone 17α-butyrate on the gonads are shown below in Table 1.

### EXPERIMENTAL SECTION

### Effect of 9,11-dehydrocortexolone on the Hypersecretion of Gonadotrophin in the Parabiotic Rat

Wistar rats of both sexes with body weights of 50-60 g were used. The males were castrated and, on the next day, were surgically united along the lateral body wall with intact females (parabiosis). In this experimental procedure, the castration of the males induces in them an immediate hypersecretion of gonadotrophin, which, on reaching the female via the parabiotic union, stimulates ovarian growth. Starting on the day after parabiosis, 9,11-dehydrocortexolone 17α-butyrate and its analogue, cortexolone 17α-propionate, were injected daily subcutaneously into the males in doses of 1 and 5 mg. Progesterone was used as a positive control and was administered by the same procedure, in doses of 0.5 and 2 mg.

Additional groups of pairs including intact males/intact females and castrated males/intact females were treated with the vehicle only and used as a control.

At the end of the treatment period, the pairs were killed and autopsies performed. The ovaries and uteruses of each female rate were isolated and weighed.

The inhibitory action causing hypersecretion of gonadotrophin due to castration was evaluated via the capacity of the tested compound to oppose the increase of weight of the ovaries.

TABLE 1
Effect of 9,11-dehydrocortexolone 17α-butyrate on the hypersecretion of
gonadotrophin in the parabiotic rat

| | Daily dose | Inhibition of ovarian |
|---|---|---|
| Treatment | (mg) | weight (%) |
| 9,11- | 1 | 59^{a} |
| dehydrocortexolone | 5 | 96^{a} |
| 17α-butyrate | | |
| cortexolone 17α- | 1 | 5 |
| propionate | 5 | 5 |
| progesterone | 0.5 | 38^{b} |
| | 2 | 66^{a} |

^{a}= P <0.01
^{b}= P <0.05 against control*
*an analysis of variance (ANOVA) was performed for each test and a value of P <0.05 was selected as the limit for statistical significance.

### Results

Table 1 above shows that 9,11-dehydrocortexolone 17α-butyrate has a strong and significant dose-correlated inhibitory effect on gonadotrophin secretion. This effect is evident from the percentage decrease in weight of the ovaries of females in parabiotic union, which decreases by 59% when 1 mg of the trial compound is administered, and by 96% when 5 mg of the same compound is administered.

The anti-gonadotrophic effect of 9,11-dehydrocortexolone 17α-butyrate is comparable to that shown by comparable doses of progesterone.

On the other hand, cortexolone 17α-propionate has no anti-gonadotrophic action at all.

1-19. (canceled)

20. A method for treating prostate tumour, polycystic ovary and/or gonadotropin-dependent precocious puberty, which comprises administering a C₃-C₁₀ 17α-ester of 9,11-dehydrocortexolone having the formula 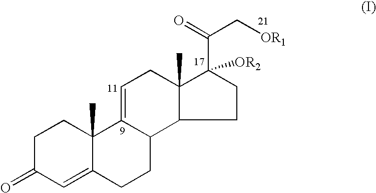
wherein R₁ is hydrogen and R₂ is C₃-C₁₀ acyl.

21. A method according to claim 20, wherein R₁ is hydrogen and R₂ is linear or branched C₄ acyl.

22. A method according to claim 20, wherein R₁ is hydrogen and R₂ is COCH₂CH₂CH₃.

23. A method according to claim 20, wherein said C₃-C₁₀ 17α-ester of 9,11-dehydrocortexolone is used for treating disorders associated with gonadotropin secretion.

24. A method according to claim 20, wherein said C₃-C₁₀ 17α-ester of 9,11-dehydrocortexolone is administered systemically.

25. A method according to claim 24, wherein said systemic way is chosen from: injectable solutions and/or suspensions, tablets, capsules, pellets oral solutions and/or suspensions, globuli, nasal sprays, vaginal suppositories, and other suppositories.

26. A method according to claim 25, wherein said C₃-C₁₀ 17α-ester of 9,11-dehydrocortexolone is administered in amounts from 0.1 to 1000 mg per unit dose.

27. A method according to claim 26, wherein said C₃-C₁₀ 17α-ester of 9,11-dehydrocortexolone is administered in amounts from 0.5 to 500 mg per unit dose.

28. A method according to claim 20, wherein said C₃-C₁₀ 17α-ester of 9,11-dehydrocortexolone is administered topically.

29. A method according to claim 28, wherein said topic way is chosen from: creams, ointments, lotions, gels, cutaneous sprays, and/or transdermal patches.

30. A method according to claim 29, wherein said C₃-C₁₀ 17α-ester of 9,11-dehydrocortexolone is administered in amounts from 0.01 to 25% by weight of the total composition.

31. A method according to claim 30, wherein said C₃-C₁₀ 17α-ester of 9,11-dehydrocortexolone is administered in amounts from 0.01 to 2% by weight of the total composition.

32. A method for controlling aberrant sexual behaviour, male contraception, hot flushes in women and/or ovarian stimulation in assisted fertilization methods, which comprises administering a C₃-C₁₀ 17α-ester of 9,11-dehydrocortexolone having the formula wherein R₁ is hydrogen and R₂ is C₃-C₁₀ acyl.

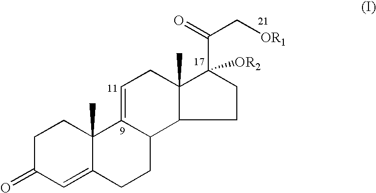

33. A method according to claim 32, wherein R₁ is hydrogen and R₂ is linear or branched C₄ acyl.

34. A method according to claim 32, wherein R₁ is hydrogen and R₂ is COCH₂CH₂CH₃.

35. A method according to claim 32, wherein said C₃-C₁₀ 17α-ester of 9,11-dehydrocortexolone is used for treating disorders associated with gonadotropin secretion.

36. A method according to claim 32, wherein said C₃-C₁₀ 17α-ester of 9,11-dehydrocortexolone is administered by systemic way.

37. A method according to claim 36, wherein said systemic way is chosen from: injectable solutions and/or suspensions, tablets, capsules, pellets oral solutions and/or suspensions, globuli, nasal sprays, vaginal suppositories, and other suppositories.

38. A method according to claim 37, wherein said C₃-C₁₀ 17α-ester of 9,11-dehydrocortexolone is administered in amounts from 0.1 to 1000 mg per unit dose.

39. A method according to claim 38, wherein said C₃-C₁₀ 17α-ester of 9,11-dehydrocortexolone is administered in amounts from 0.5 to 500 mg per unit dose.

40. A method according to claim 32, wherein said C₃-C₁₀ 17α-ester of 9,11-dehydrocortexolone is administered topically.

41. A method according to claim 40, wherein said topic way is chosen from: creams, ointments, lotions, gels, cutaneous sprays, and/or transdermal patches.

42. A method according to claim 41, wherein said C₃-C₁₀ 17α-ester of 9,11-dehydrocortexolone is administered in amounts from 0.01 to 25% by weight of the total composition.

43. A method according to claim 42, wherein said C₃-C₁₀ 17α-ester of 9,11-dehydrocortexolone is administered in amounts from 0.01 to 2% by weight of the total composition.

44. Pharmaceutical compositions containing as their active principle C₃-C₁₀ 17α-esters of 9,11-dehydrocortexolone, having the formula 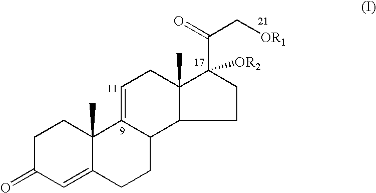
in which R₁ is hydrogen and R₂ is C₃-C₁₀ acyl, in association with pharmacologically acceptable excipients.

45. Pharmaceutical compositions according to claim 44, wherein R₁ is hydrogen and R₂ is linear or branched C₄ acyl.

46. Pharmaceutical compositions according to claim 44, wherein R₁ is hydrogen and R₂ is COCH₂CH₂CH₃.

47. Pharmaceutical compositions according to claim 44, for the treatment of disorders associated with gonadotropin secretion.

48. Pharmaceutical compositions according to claim 47, wherein said disorders are chosen from: prostate tumour, polycystic ovary, gonadotropin-dependent precocious puberty, control of aberrant sexual behaviour, male contraception, hot flushes in women and/or in ovarian stimulation in assisted fertilization methods.

49. Pharmaceutical compositions according to claim 44, in the form of injectable solutions and/or suspensions, tablets, capsules, pellets oral solutions and/or suspensions, transdermal patches, suppositories, globuli, vaginal suppositories, creams, ointments, lotions, cutaneous and/or nasal sprays and/or gels.
